(19) United States
(12) Patent Application Publication
(10) Pub. No.: US 2009/0214516 A1
(43) Pub. Date: Aug. 27, 2009

(54) Inhibitors of Reglll Proteins as Asthma Therapeutics

(76) Inventors: Maximillian T. Follettie, Belmont, MA (US); Debra D. Donaldson, Medford, MA (US)

Correspondence Address: WilmerHale/Wyeth, 60 STATE STREET, BOSTON, MA, 02109, US

(21) Appl. No.: 11/628,255

(22) PCT Filed: Jun. 3, 2005
(86) PCT No.: PCT/US05/19594

§ 371 Date: May 22, 2007

Related U.S. Application Data
(60) Provisional application No. 60/577,258, filed on Jun. 4, 2004.

(51) Int. Cl. A61K 39/395; G01N 33/53; C12Q 1/68; A61K 31/7088

(52) U.S. Cl. 424/130.1; 435/7.1; 435/6; 514/44 R

(57) ABSTRACT

Methods of screening for agents for treating asthma are provided. The methods involve screening for agents that decrease the production or activity of a RegIII protein that has been discovered herein to play a role in producing the symptoms and pathological complications involved in asthma. Methods of treating asthma, as well as screening for and treating with inhibitors of a RegIII protein are also provided.

### FIELD OF THE INVENTION

It is an object of the invention to provide methods of screening for agents for treating asthma. It is a further object of the invention to provide methods for treating asthma. These and other objects and advantages of the present invention will be apparent from the descriptions herein.

### BACKGROUND OF THE INVENTION

The present invention relates generally to asthma therapeutics. Specifically, the invention relates to methods of screening for agents for treating asthma and methods for treating asthma.

Asthma is a chronic inflammatory disease of the airways characterized by recurrent episodes of reversible airway obstruction and airway hyperresponsiveness (AHR). Typical clinical manifestations include shortness of breath, wheezing, coughing and chest tightness that can become life threatening or fatal. While existing therapies focus on reducing the symptomatic bronchospasm and pulmonary inflammation, there is a growing awareness of the role of long term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity, and mortality of the disease has increased during the past two decades. In 2000, in the United States alone, nearly 1.8 million emergency room visits, 465,000 hospitalizations and 4,487 deaths were directly attributed to asthma. Asthma-related healthcare costs are estimated at $14 billion annually.

It is generally accepted that allergic asthma is initiated by an inappropriate inflammatory reaction to airborne allergens. The lungs of asthmatics demonstrate an intense infiltration of lymphocytes, mast cells and especially eosinophils. A large body of evidence has demonstrated this immune response is driven by CD4⁺ T-cells expressing a T_{H}2 cytokine profile. One murine model of asthma involves sensitization of the animal to ovalbumin (OVA) followed by intratracheal delivery of the OVA challenge. This procedure generates a T_{H}2 immune reaction in the mouse lung and mimics four major pathophysiological responses seen in human asthma, including upregulated serum IgE (atopy), eosinophilia, excessive mucus secretion, and AHR. The cytokine IL-13, expressed by basophils, mast cells, activated T cells and NK cells, plays a central role in the inflammatory response to OVA in mouse lungs. Direct lung instillation of murine IL-13 elicits all four of the asthma-related pathologies and, conversely, the presence of a soluble IL-13 antagonist (sIL-13Rα2-Fc) completely blocked both the OVA-challenge induced goblet cell mucus synthesis and the AHR to acetylcholine. Wills-Karp, M., et al., “Interleukin-13: central mediator of allergic asthma,” Science 282(5397): 2258-2261 (1998); Grunig, G., et al., “Requirement for IL-13 independently of IL-4 in experimental asthma,” Science 282(5397): 2261-2263 (1998). Thus, IL-13 mediated signaling is sufficient to elicit all four asthma-related pathophysiological phenotypes and is required for the hypersecretion of mucus and induced AHR in the mouse model.

Biologically active IL-13 binds specifically to a low-affinity binding chain IL-13Rα1 and to a high-affinity multimeric complex composed of IL-13Rα1 and IL-4R, a shared component of IL-4 signaling complex. Wills-Karp, M., “IL-12/IL-13 axis in allergic asthma,” J Allergy Clin Immunol 107(1): 9-18 (2001). Activation of the IL-13 pathway cascade triggers the recruitment, phosphorylation and ultimate nuclear translocation of the transcriptional activator Stat6. A number of physiological studies demonstrate the inability of pulmonary OVA-challenge to elicit major pathology related phenotypes including eosinophil infiltration, mucus hypersecretion and airway hyperreactivity in mice homozygous for the Stat6^{-/-} null allele. Kuperman, D., et al., “Signal transducer and activator of transcription factor 6 (Stat6)-deficient mice are protected from antigen-induced airway hyperresponsiveness and mucus production,” J Exp Med 187(6): 939-48 (1998). Recent genetic studies have demonstrated a linkage between specific human alleles of IL-13 and its signaling components with asthma and atopy, demonstrating the critical role of this pathway in the human disease. Shirakawa et al., “Atopy and asthma: genetic variants of IL-4 and IL-13 signaling,” Immunol. Today 21(2):60-64 (2000).

IL-13 also binds to an additional receptor chain, IL-13Rα2, expressed in both human and mouse with as yet undefined biological function. The murine IL-13Rα2 binds IL-13 with approximately 100-fold greater affinity (Kd of 0.5 to 1.2 nM) relative to IL-13Rα1, allowing the construction of a potent soluble IL-13 antagonist, sIL-13Rα2-Fc. The sIL-13Rα2-Fc has been used as an antagonist in a variety of disease models to demonstrate the role of IL-13 in Schistosomiasis induced liver fibrosis and granuloma formation, tumor immune surveillance, as well as in the OVA-challenge asthma model. Wills-Karp, M., et al., “Interleukin-13: central mediator of allergic asthma,” Science 282(5397): 2258-2261 (1998); Grunig, G., et al., “Requirement for IL-13 independently of IL-4 in experimental asthma,” Science 282(5397): 2261-2263 (1998).

RegIII protein is a subclass of the Reg gene family, a multi-gene family that also includes RegI, which is a growth factor for β-cells, and RegII. Okamoto, H., J. Hepatobiliary Pancreat. Surg. 6:254-262 (1999). In humans, the RegIII family includes HIP, a gene expressed in hepatocellular carcinoma, intestine, and pancreas, and pancreatitis-associated protein (PAP). Id. Other REG family genes that have been found in humans are REGIα, REGIβ, and REG-related sequence. Id. Mouse REG genes include RegI, RegII, RegIIIα, RegIIIβ, RegIIIγ, and RegIIIδ. Id. REG genes have also been observed in rat, cow and hamster. Id. Due to the mitogenic activities of other Reg family member cells and the homology between family members, it is believed that Reg proteins are growth factors. Id. Because of the degree of sequence homology between the members of the RegIII subclass, the inventors believe that these genes and proteins are related in biological structure and function.

Current therapy of asthma to treat bronchospasms and airway inflammation includes use of bronchodilators, corticosteroids, and leukotriene inhibitors. Many of such treatments include undesired side effects and lose effectiveness after being used for a period of time. Additionally, limited agents for therapeutic intervention are available that decrease the airway remodeling process that occurs in asthmatics. Therefore, there remains a need for an increased molecular understanding of asthma, coupled to identification of novel therapeutic strategies to combat this complex disease. The present invention addresses these needs.

### SUMMARY OF THE INVENTION

It has been discovered that the messenger RNA (mRNA) of a RegIII protein is statistically significantly increased in an animal model of asthma compared to control, non-asthmatic animals. Specifically, the mRNA encoding a RegIII protein has been found to be elevated by either intratracheal ovalbumin challenge or direct pulmonary instillation of IL-13 and has herein been discovered as a target for asthma therapeutics. Accordingly, in one aspect of the invention, methods of screening for agents for treating asthma are provided. Methods for treating asthma are also provided.

In one aspect of the invention, a method of screening for agents for treating asthma includes (a) contacting a RegIII protein with a test agent; and (b) determining if the test agent inhibits the activity of the RegIII protein, wherein a test agent that inactivates the activity of the RegIII protein is useful for treating asthma. In some aspects, the method further includes a step of classifying the test agent as an agent for treating asthma if it inhibits the activity of the RegIII protein.

In another aspect, the invention provides a method of screening for agents for treating asthma by (a) contacting a nucleotide sequence encoding a reporter gene product operably linked to a RegIII protein promoter with a test agent; and (b) determining if the test agent inhibits production of the reporter gene product, wherein a test agent that inhibits production of the reporter gene product is useful for treating asthma. In some aspects, the method further includes a step of classifying the test agent as an agent for treating asthma if it inhibits production of the reporter gene product.

In yet another aspect of the invention, methods for treating asthma are provided. In one embodiment, a method includes administering to a mammal in need thereof a therapeutic amount of an agent that decreases the activity of a RegIII protein. In a further embodiment of the invention, a method includes administering to a mammal in need thereof a therapeutic amount of an agent that decreases the production of a RegIII protein.

### DESCRIPTION OF THE FIGURES

FIG. 1 is a representation of the mRNA sequence of human pancreatitis-associated protein (PAP), which is a member of the RegIII family (SEQ ID NO:1) as reported in Dusetti, N.J., et al., Genomics 19(1):108-114 (1994). Residues 33-557 represent the coding sequence (Genbank accession number NP_{—}002571).

FIG. 2 is a representation of the amino acid sequence of the translated human PAP (SEQ ID NO:2) as reported in Dusetti et al., supra (Genbank accession number NM_{—}002580).

FIG. 3 is a representation of the mRNA sequence of murine (Mus musculus) RegIIIγ protein (SEQ ID NO:3) as reported in Narushima Y., et al., Gene 185(2):159-68 (Feb. 7, 1997), where residues 33 to 557 represent the coding sequence (Genbank accession number D63361).

FIG. 4 is a representation of the amino acid sequence the translated murine (Mus musculus) RegIIIγ protein (SEQ ID NO:4) as reported in Narushima et al., supra.

FIG. 5 (SEQ ID NO:5) is a representation of the nucleotide sequence of the promoter for human PAP.

FIG. 6 (SEQ ID NO:6) is a representation of the nucleotide sequence of the promoter for mouse RegIIIγ protein.

### DETAILED DESCRIPTION OF THE INVENTION

The patent and scientific literature referred to herein establishes knowledge that is available to those of skill in the art. The issued U.S. patents, allowed applications, published applications (U.S. and foreign) and references, including GenBank database sequences, that are cited herein are incorporated by reference to the same extent as if each was specifically and individually indicated to be incorporated by reference.

The invention is based upon the unexpected discovery that the mRNA of a RegIII protein is significantly increased in an animal model of asthma compared to control, non-asthmatic animals. Specifically, the mRNA encoding RegIIIγ protein is elevated by either intratracheal ovalbumin challenge or direct pulmonary instillation of IL-13. Moreover, blocking the ability of IL-13 to bind to the IL-13 receptor in turn inhibits expression of RegIIIγ. The upregulation of RegIIIγ protein in murine asthma models indicates that Reg proteins are involved in the allergic response and also that RegIII proteins are regulated through the IL-13 pathway.

Reg proteins comprise a multigene family, grouped into three subclasses based on sequence homology. Best characterized is RegI, originally identified as a growth factor in pancreatic [symbol] -cell regeneration and its expression is upregulated in wound healing models of both regenerating pancreas and healing gastric mucosa. RegI is mitogenic to gastric epithelial cells and induces the in vivo proliferation of pancreatic [symbol] cells in depancreatized rats. RegIII, part of a Reg multigene complex on mouse chromosome 6, is almost exclusively expressed in the intestine and colon. Pulmonary IL-13 administration, either by direct instillation or by transgenic lung-specific expression of IL-13, has been shown to generate airway epithelial cell hypertrophy. The mitogenic properties of closely related Reg proteins make the RegIII family candidate genes for further investigation into the pulmonary epithelial hypertrophy observed in various animal disease models and human asthma. The inventors believe that RegIII proteins stimulate epithelial growth and/or hypertrophy in, for example, the lungs. Epithelial cell thickening observed in asthma from epithelial cell growth and/or hypertrophy is part of the remodeling process in the lung. RegIII proteins are identified herein as target genes to combat pulmonary epithelial hypertrophy and airway remodeling processes observed in human asthma due to the mitogenic properties of Reg proteins and the upregulation of the mRNA of RegIII family proteins in mouse allergic asthma models discussed herein. RegIII family proteins include, without limitation human HIP, human PAP, mouse RegIIIα, mouse IIIβ, mouse IIIγ, mouse IIIδ, rat PAP, rat PAP III, bovine PTP, and hamster islet neogenesis-associated protein (INGAP). Thus, the inventors believe that the proteins of the RegIII family are involved in the allergic response in asthma, and, consequently, that an inhibitor of the Reg proteins will be effective in treating asthma. “Asthma”, as used herein includes, but is not limited to, atopic asthma, nonatopic asthma, allergic asthma, exercise-induced asthma, drug-induced asthma, occupational asthma and late stage asthma.

As noted above, the invention provides methods of screening for agents for treating asthma in a mammal. In one embodiment, the mammal is a human. As used herein, “agent” includes, but is not limited to, synthetic small molecules, chemicals, nucleic acids such as antisense oligonucleotides, RNA, inhibitors such as siRNA, ribozymes, nucleic acid ligands such as aptamers, peptides and proteins such as hormones, cytokines, antibodies and portions thereof. In one aspect, the methods include contacting a RegIII protein with a test agent and a substrate of the protein. In one aspect, the test agent modulates (e.g., inhibits or increases) the activity of a RegIII protein. In another aspect, the test agent modulates (e.g., inhibits or increases) the production or expression of a RegIII protein. A “test agent” is a putative “agent,” the modulating ability of which has not yet been confirmed. Once test agents are screened, they are classified as “agents,” if they are shown to modulate protein activity or production or expression (for example by modulating transcription or translation). In a particular embodiment, the agent reduces or inhibits the activity of a RegIII protein.

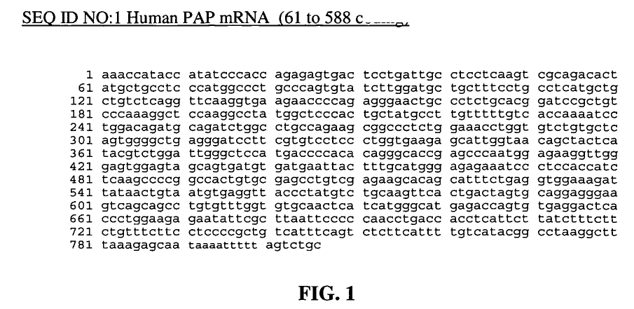

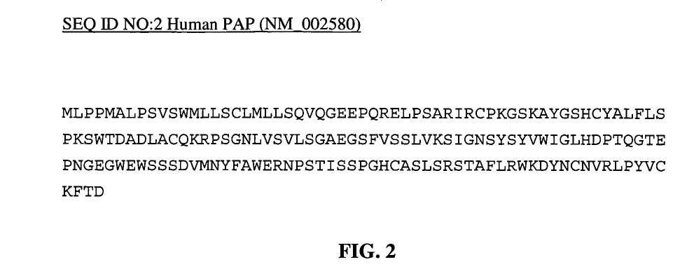

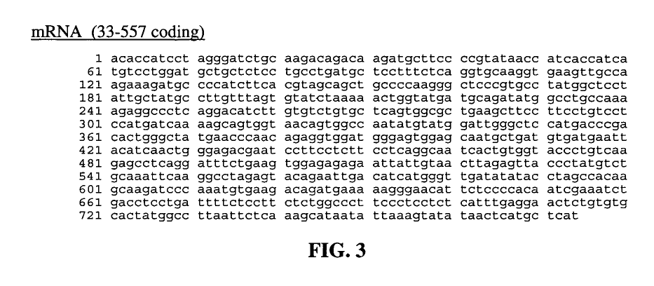

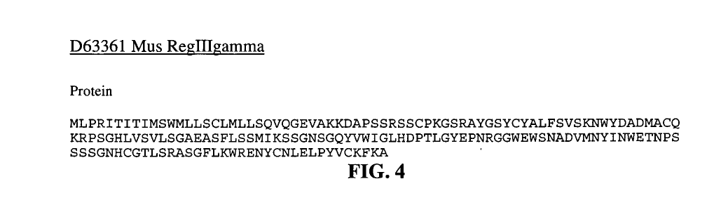

In some embodiments the agent binds to a RegIII protein. In other embodiments, the agent interacts with (such as by chemically modifying) an inhibitor or activator of a RegIII protein activity or expression. By way of a nonlimiting example, an agent may bind to and inhibit an activator of a RegIII protein or an agent may bind to and activate an inhibitor of the activity of a RegIII protein. Moreover, in another nonlimiting example, the agent may interact upstream of a RegIII protein, such as by antagonizing IL-13, the IL-13 receptor, or the IL-4 receptor. The methods include: determining if the test agent modulates (e.g., inhibits) the activity of a RegIII protein and classifying the test agent as an agent for treating asthma if the test agent modulates (e.g., inhibits) the activity or the expression of the protein. The nucleotide and amino acid sequences of human PAP, which is a member of the RegIII protein family, are set forth in SEQ ID NO:1 and SEQ ID NO:2, as provided in FIGS. 1 and 2, respectively. The nucleotide and amino acid sequences of murine RegIIIγ protein are set forth in SEQ ID NO:3 and SEQ ID NO:4, as provided in FIGS. 3 and 4, respectively. Exemplary agents that inhibit the activity or expression of RegIII proteins include, without limitation, antagonists of IL-13 such as sIL-13Rα1-Fc, sIL-13Rα2-Fc, antagonists of the IL-13 receptor, antagonists of the IL-4 receptor, antibodies such as anti-RegIII protein antibodies (e.g., anti-HIP, anti-PAP, anti-RegIIIγ), IL-13 neutralizing antibodies, IL-13R antibodies, nucleic acids such as siRNA, aptamers (i.e., nucleic acid ligands), antisense oligonucleotides and ribozymes and small molecule chemical inhibitors. In the case of antibodies, the method includes, without limitation, a polyclonal antibody, a monoclonal antibody, a chimeric antibody, a humanized antibody, a genetically engineered antibody, a bispecific antibody, antibody fragments (including but not limited to “Fv,” “F(ab′)2,” “F(ab),” and “Dab”) and single chains representing the reactive portion of the antibody. Such an antibody includes antibodies belonging to any of the immunoglobulin classes, such as IgM, IgG, IgD, IgE, IgA or their subclasses or mixtures thereof. The invention further includes derivatives of these antibodies, such as those that retain their binding activity to the RegIII protein while altering one or more other properties related to their use as a pharmaceutical agent, e.g., serum stability or efficiency of production.

In another embodiment, the production of a RegIII protein is inhibited. The methods include, without limitation, determining if the test agent modulates (e.g., inhibits) the expression of a RegIII protein and classifying the test agent as an agent for treating asthma if the test agent modulates (e.g., inhibits) the expression of the RegIII protein. Exemplary agents that inhibit the expression of RegIII proteins include, without limitation, antagonists selected from the group consisting of soluble IL-13 receptors (for example, extracellular domains of IL-13Rα1 and IL-13Rα2, alone or fused to a heterologous moiety, for example, Fc, such as 13RαsIL-13Rα1-Fc, sIL-13Rα2-Fc); neutralizing antibodies against IL-13 or IL-13R or antigen-binding fragments thereof; ribozymes; antisense oligonucleotides; RNA inhibitors (for example, siRNA); hormones; cytokines; and chemicals (for example, small molecules). See Wynn et al. U.S. Pat. No. 6,664,227, which is herein incorporated in its entirety by reference.

The discovery that RegIII proteins are associated with inducing the symptoms and/or complications of asthma renders the sequences of RegIII proteins useful in methods of identifying agents of the invention. Such methods include assaying potential agents for the ability to inhibit the activity and/or production or expression of a RegIII protein. Polynucleotides and polypeptides useful in these assays include not only the genes and encoded polypeptides disclosed herein, but also variants thereof that have substantially the same activity as wild-type genes and polypeptides. “Variants” as used herein, includes polynucleotides or polypeptides containing one or more deletions, insertions or substitutions, as long as the variant retains substantially the same activity of the wild-type polynucleotide or polypeptide. With regard to polypeptides, deletion variants are contemplated to include fragments lacking portions of the polypeptide not essential for biological activity, and insertion variants are contemplated to include fusion polypeptides in which the wild-type polypeptide or fragment thereof has been fused to another polypeptide.

Accordingly, in one embodiment, a RegIII protein utilized in the invention may be encoded by a nucleotide sequence that has at least about 60%, at least about 70%, at least about 80% or at least about 90% identity to the nucleotide sequence set forth in SEQ ID NO:1 (FIG. 1) or SEQ ID NO:3 (FIG. 3). Percent identity may be determined, for example, by comparing sequence information using the advanced BLAST computer program, version 2.0.8, available from the National Institutes of Health.

Additionally, in another embodiment, a RegIII protein may be encoded by nucleotide sequences having substantial similarity to the nucleotide sequence set forth in SEQ ID NO:1 (FIG. 1) or SEQ ID NO:3 (FIG. 3). “Substantial similarity,” as used herein means that the nucleotide sequence is sufficiently similar to a reference nucleotide sequence that it will hybridize therewith under moderately stringent conditions. This method of determining similarity is well known in the art to which the invention pertains. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| Strin- | Poly- | Hybrid | Hybridization | Wash |
|---|---|---|---|---|
| gency | nucleotide | Length | Temperature and | Temperature |
| Condition | Hybrid | (bp)¹ | Buffer² | and Buffer² |
| A | DNA:DNA | >50 | 65° C.; 1X SSC -or- | 65° C.; 0.3X |
| | | | 42° C.; 1X SSC, | SSC |
| | | | 50% formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X |
| | | | 45° C.; 1X SSC, | SSC |
| | | | 50% formamide | |
| D | DNA:RNA | <50 | T_{D}*; 1X SSC | T_{D}*; 1X SSC |
| E | RNA:RNA | >50 | 70° C.; 1X SSC -or- | 70° C.; |
| | | | 50° C.; 1X SSC, | 0.3xSSC |
| | | | 50% formamide | |
| F | RNA:RNA | <50 | T_{F}*; 1X SSC | T_{f}*; 1X SSC |
| G | DNA:DNA | >50 | 65° C.; 4X SSC -or- | 65° C.; 1X |
| | | | 42° C.; 4X SSC, | SSC |
| | | | 50% formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X |
| | | | 45° C.; 4X SSC, | SSC |
| | | | 50% formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X |
| | | | 50° C.; 4X SSC, | SSC |
| | | | 50% formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X |
| | | | 40° C.; 6X SSC, | SSC |
| | | | 50% formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X |
| | | | 42° C.; 6X SSC, | SSC |
| | | | 50% formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X |
| | | | 45° C.; 6X SSC, | SSC |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T bases)+ 4(# of G + C bases). For hybrids between 18 and 49 base pairs in length, Tₘ(EC) = 81.5 + 16.6(log₁₀Na⁺) + 0.41(% G + C) − (600/N), where N is the number of bases in the hybrid, and Na+ is the concentration of sodium ions in the hybridization buffer (Na+ for 1xSSC = 0.165 M).

Additional examples of stringency conditions for polynucleotide hybridization are provided in Sambrook et al., “Molecular Cloning: A Laboratory Manual”, Chs. 9 & 11, Cold Spring Harbor Laboratory Press, Cold Spring Harbor, N.Y. (1989), and Ausubel et al., eds., Current Protocols in Molecular Biology, §§ 2.10, 6.3-6.4, John Wiley & Sons, Inc. (1995), herein incorporated by reference.

RegIII proteins may be produced by methods known to the skilled artisan. For example, a nucleotide sequence encoding a RegIII protein gene may be introduced into a desired host cell. Such a nucleotide sequence may first be inserted into an appropriate recombinant expression vector.

Recombinant expression vectors may be constructed by incorporating the above-recited nucleotide sequences within a vector according to methods well known to the skilled artisan. A wide variety of vectors are known that are useful in the invention. Suitable vectors include plasmid vectors and viral vectors, including retrovirus vectors, adenovirus vectors, adeno-associated virus vectors and herpes viral vectors. The vectors may include other known genetic elements necessary or desirable for efficient expression of the nucleic acid in a specified host cell, including regulatory elements. For example, the vectors may include a promoter and any necessary enhancer sequences that cooperate with the promoter to achieve transcription of the gene. The nucleotide sequence may be operably linked to such regulatory elements.

Such a nucleotide sequence is referred to as a “genetic construct.” A genetic construct may contain a genetic element on its own or in combination with one or more additional genetic elements, including but not limited to genes, promoters, or enhancers. In some embodiments, these genetic elements are operably linked. In some embodiments, the specific gene at issue (e.g., hHIP, hPAP, RegIIIγ) may not be present in the genetic construct, including, but not limited to, a situation in which a promoter of a RegIII protein is operably linked to a reporter gene.

As used herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. “Operably linked” means that the DNA sequences being linked are typically contiguous and, where necessary to join two protein coding regions, contiguous and in reading frame. However, since enhancers may function when separated from the promoter by several kilobases and intron sequences may be of variable lengths, some nucleotide sequences may be operably linked but not contiguous

A wide variety of methods are available for introducing the nucleotide sequence encoding RegIII proteins, and which may be included in a recombinant expression vector, into a host cell. Such methods are known to the art and include mechanical methods, chemical methods, lipophilic methods and electroporation. Microinjection and use of a gene gun with, for example, a gold particle substrate for the DNA to be introduced is a representative, non-limiting exemplary mechanical method. Use of calcium phosphate or DEAE-Dextran is a representative, non-limiting exemplary chemical method. Exemplary lipophilic methods include use of liposomes and other cationic agents for lipid-mediated transfection. Such methods are well known to the art.

A wide variety of host cells may be utilized in the present invention to produce the desired quantities of RegIII protein. Such cells include, but are not limited to, eukaryotic and prokaryotic cells, including mammalian cells and bacterial cells known to the art.

RegIII protein may be isolated and purified by techniques well known to the skilled artisan, including, but not limited to, chromatographic, electrophoretic and centrifugation techniques. Such methods are known to the art.

The sample (e.g., tissue, cell culture, or amount of a RegIII protein) is typically contacted with a test agent for a time period sufficient to inhibit the activity or production/expression of the protein. This time period may vary depending on the nature of the test agent, the particular RegIII protein, the activity or expression detection method selected, and the sample tissue selected. The skilled artisan without undue experimentation may readily determine such times. An exemplary test agent is one that binds to or otherwise decreases the activity of the protein by, for example, binding to a RegIII protein, blocking its ability to functionally interact with other components of the signal transduction pathway. Similar assays screen for test agents that block the expression of a RegIII protein, including without limitation such as IL-13 antagonists (e.g., sIL-13Rα2-Fc fusion proteins) or IL-4 antagonists.

A wide variety of assays may be utilized to determine whether the test agent inhibits the activity of a RegIII protein. As the RegIII proteins have mitogenic activity, such assays include without limitation cell proliferation assays and mucus production assays. In one embodiment, desired cells are contacted or otherwise incubated with an effective amount of a RegIII protein and the test agent. This amount is effective for stimulating epithelial cell growth and/or hypertrophy and can be determined by the skilled artisan. Cellular proliferation is then measured and may be compared to control cells treated with the RegIII protein in the absence of the test agent

A wide variety of cellular proliferation assays may be utilized to determine whether the test agent inhibits the activity of a RegIII protein. Such cell proliferation assays are widely known in the art, and include, for example a labeled thymidine uptake assay, a 5-bromo-2′-deoxyuridine uptake assay, a 3-[4,5-dimethylthiazol-2-yl]-2,5-diphenyltetrasodium bromide assay, an adenosine triphosphate generation assay, or some combination thereof.

As known in the art, labeled thymidine incorporation, such as radioactively-labeled thymidine incorporation, may be used to monitor or otherwise quantify cellular proliferation. Cells incorporate the labeled nucleotide, typically tritiated thymidine, into newly synthesized DNA. The relative amount of cellular proliferation is determined by quantitating the amount of radioactively labeled nucleotide incorporated into the DNA. Cunningham, B. A., The Scientist, 15(13):26 (2001). The amount of the radioactively labeled nucleotide incorporated into DNA may be measured by liquid scintillation counting and is a measure of cellular proliferation.

Other nucleotides may similarly be incorporated into newly synthesized DNA, quantitated and act as a relative measure of cellular proliferation. For example, 5-bromo-2′-deoxyuridine (BrdU) may be incorporated into DNA and may be quantitated by using commercially available monoclonal antibodies against BrdU in an enzyme immunoassay, such as an enzyme-linked immunosorbent assay (ELISA).

The 3-[4,5-dimethylthiazol-2-yl]-2,5-diphenyltetrasodium bromide (MTT) assay involves diminution of cellular reduction of MTT to a blue compound called formazan. The cellular reduction is increased in actively-proliferating cells relative to senescent or dying cells. Thus, quantitation of formazan by observing the intensity of the blue color (e.g., spectroscopically) provides a relative measure of cellular proliferation. Cunningham, B. A., The Scientist, 15(13):26 (2001); Mosmann, J. Immunol. Methods 65:55-63 (1983). Other tetrazolium salts may be utilized instead of or in addition to MTT, including WST-1, WST-8, XTT, and MTS. Kits are commercially available for performing such assays, including from American Type Culture Collection (Manassas, Va.).

The adenosine triphosphate generation assay measures the amount of ATP present in cells being studied. ATP is typically reacted with various components which hydrolyze ATP, resulting in the production of light. The intensity of the light is proportional to the amount of cellular ATP. In one assay, ATP is reacted with luciferase and luciferin in the presence of oxygen. Such assays are described in, for example, The Scientist, 15(13):26 (2001); Mosmann, J. Immunol. Methods 65:55-63 (1983) and Crouch et al., J. Immunol. Methods 160:81-88 (1993).

Other methods of quantitating cells may be utilized that are known to the art, including use of a hemacytometer and trypan blue staining as described, for example, in Ausubel, et al., eds., Current Protocols in Molecular Biology, John Wiley & Sons.

A wide variety of cells may be utilized in the cellular proliferation assay. Nonlimiting examples of cells are mammalian cells and are responsive to the mitogen activity of the RegIII proteins. Such cells include, for example, primary epithelial cells and or cell lines, such as LIM1863 (Whitehead et al. Cancer Res. 47(10):2683-9 (May 15, 1987)). Such cells are typically present as a cell culture. Other nonlimiting examples of cells are airway epithelial cells. Cell culture methods are known to the skilled artisan, and are described in, for example, Ausubel et al., eds., Current Protocols in Molecular Biology, John Wiley & Sons; A. J. Shaw, ed., Epithelial Cell Culture: A Practice Approach (Practical Approach Series), IRL press, 1996; R. I. Freshney, and M. G. Freshney, eds., Culture of Epithelial Cells, 2nd Edition, John Wiley & Sons, 2002; and C. Wise, ed., Epithelial Cell Culture Protocols (Methods in Molecular Biology, v. 188), Humana Press, 2002.

Another assay that may be utilized to determine whether the test agent inhibits the activity of a RegIII protein is quantitation of mucus produced from the cells stained with H&E, a technique known to those of skill in the art. In one embodiment, epithelial cells are contacted or otherwise incubated with an effective amount of a RegIII protein and the test agent. This amount is effective for stimulating differentiation of the epithelial cell cells into goblet cells and can be determined by the skilled artisan. Mucus production may then be measured and may be compared to control cells treated with a RegIII protein in the absence of the test agent. In addition to H&E staining for mucus production, decreases in mucus encoding mRNA expression can be measured using classical reverse transcriptase polymerase chain reaction (RT-PCR), global profiling of expressed mRNA using cDNA or oligonucleotide arrays, or quantitating the amount of mucopolysaccharides in the mucus.

A wide variety of assays are available for quantitating mucus, including use of stains to stain mucosal components and quantitation by a colorimetric assay. In one embodiment, a periodic acid Schiff technique may be used to stain mucins, glycoproteins or mucopolysaccharides present in mucus. Alternatively, mucins may be radiolabelled and analyzed by hydrophobic interaction chromatography as described in Svitacheva, N. and Davies, J. R., “Mucin biosynthesis and secretion in tracheal epithelial cells in primary culture,” Biochem. J. 353:23-32 (2001). The mucus may be isolated by standard methods known to the art and as described, for example, in Svitacheva, N. and Davies, J. R., supra. Briefly, cells in culture may be harvested and the mucins may be isolated by isopycnic density-gradient centrifugation after dialyzing the samples.

Mammalian epithelial cells from a wide variety of sources may be utilized in the above-referenced screening assays involving quantitation of mucin, as well as for the cellular proliferation assays described above. Nonlimiting examples of epithelial cells are small airway epithelial cells or bronchial epithelial cells, which can be obtained from Clonetics (www.cambrex.com). Such cells are typically present as a cell culture. Cell culture methods are known to the skilled artisan, and described in, for example, Ausubel et al., eds., Current Protocols in Molecular Biology, John Wiley & Sons; A. J. Shaw, ed., Epithelial Cell Culture: A Practice Approach (Practical Approach Series), IRL press, 1996; R. I. Freshney, and M. G. Freshney, eds., Culture of Epithelial Cells, 2nd Edition, John Wiley & Sons, 2002; and C. Wise, ed., Epithelial Cell Culture Protocols (Methods in Molecular Biology, v. 188), Humana Press, 2002. In addition, epithelial cell lines may be used in screening assays involving the quantitation of mucin.

A wide variety of test agents may be tested in the screening methods of the present invention. For example, small molecule compounds, known in the art, synthetic small molecule chemicals, nucleic acids such as antisense oligonucleotides, RNA inhibitors such as siRNA, ribozymes, and aptamers, peptides and proteins such as hormones, antibodies, and portions thereof, may act as test agents. In one nonlimiting example, the three-dimensional structure of the active site of a RegIII protein is determined by crystallizing the complex formed by the enzyme and a known inhibitor. Rational drug design is then used to identify new test agents by making alterations in the structure of a known inhibitor or by designing small molecule compounds that bind to the active site of the enzyme. Similarly, the skilled artisan would recognize that rational drug design could also be used to design antagonists of IL-13R and/or IL-4R, which would also be useful in modulating the production of a RegIII protein. As discussed elsewhere herein, test agents include inhibitors of the activity of a RegIII protein as well as inhibitors of the expression or production of a RegIII protein. Nonlimiting examples of inhibitors of RegIII activity include antibodies to RegIII proteins, small molecule inhibitors, and proteins and peptides such as hormones and cytokines. Inhibitors of RegIII production include, without limitation, test agents that inhibit the production of a RegIII mRNA or protein. Nonlimiting examples of such test agents include antagonists of IL-13, siRNA, antisense nucleic acids, ribozymes, aptamers, neutralizing antibodies to IL-13, IL-13R and IL-4R. Antagonists of IL-13 include without limitation test agents that block the ability of IL-13 to bind to either the IL-13 receptor or the IL-4 receptor. Such antagonists include without limitation IL-13Rα1-Fc, sIL-13Rα2-Fc, and neutralizing antibodies against IL-13, IL-13R or IL-4R.

In one embodiment, the invention also provides a method of screening for agents for treating asthma in a mammal by screening for an agent that modulates (e.g., inhibits or activates) the activity or production or expression of a RegIII protein. The method includes contacting a nucleotide sequence encoding a reporter gene product operably linked to a promoter of a mammalian gene encoding a RegIII protein, including without limitation hHIP, hPAP, or RegIIIγ, with a test agent thought to be effective in inhibiting production of a RegIII protein; determining if the test agent inhibits production of the reporter gene product; and classifying the test agent as an agent for treating asthma if the test agent inhibits production of the reporter gene product. In one embodiment, the mammal is a human.

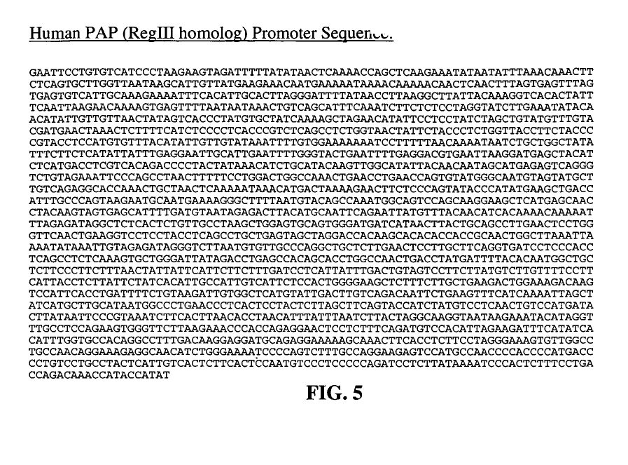

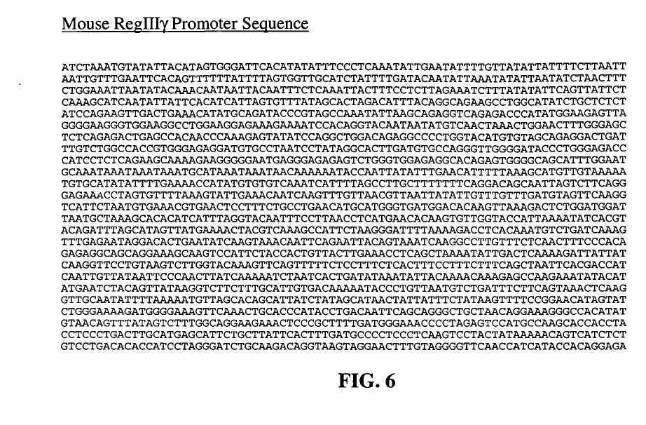

In one embodiment, the promoter of the gene encoding a RegIII protein, including without limitation hHIP, hPAP, or RegIIIγ, includes a nucleotide sequence set forth in SEQ ID NO:5 or SEQ ID NO:6, as set forth in FIGS. 5 and 6, respectively. Nucleotide sequences having at least about 50%, at least about 70%, at least about 80% and at least about 90% identity to such sequences and that function as promoter, for example, to direct expression of a gene encoding a RegIII protein described herein, are also encompassed in the invention.

The nucleotide sequence of promoters of RegIII proteins is determined by art-recognized methods. One nonlimiting example of such a method is to screen a genomic library (e.g., a YAC human genomic library) for the promoter sequence of interest using SEQ ID NO:1 (FIG. 1) or SEQ ID NO:3 (FIG. 3) as a probe. Another nonlimiting example of a method to determine the appropriate promoter sequence is to perform a Southern blot of the human genomic DNA by probing electrophoretically resolved human genomic DNA with a probe (e.g., a probe comprising SEQ ID NO:1 or a portion thereof) and then determining where the cDNA probe (e.g., SEQ ID NO:1) hybridizes. Upon determining the band to which the probe (e.g., SEQ ID NO:1) hybridizes, the band can be isolated (e.g., cut out of the gel) and subjected to sequence analysis. This allows detection of the nucleotide fragment 5′ of nucleotides 104-106 (i.e., the ATG site) of SEQ ID NO:1. The nucleotide fragment may be between approximately 500 to 1000 units in length. The promoter sequence for murine RegIIIγ protein set forth in SEQ ID NO:3 (FIG. 3) may be determined by these methods as well. Nucleotide sequences having at least about 70%, at least about 80% and at least about 90% identity to such sequences and that function as promoter, for example, to direct expression of a gene encoding a RegIII protein described herein, are also encompassed in the invention.

A wide variety of reporter genes may be operably linked to the RegIII protein promoter described above. Such genes may encode, for example, luciferase, β-galactosidase, chloramphenical acetyltransferase, β-glucuronidase, alkaline phosphatase, and green fluorescent protein, or other reporter gene products known to the art.

In an embodiment of the invention, the nucleotide sequence encoding a reporter gene that is operably linked to a RegIII protein promoter is introduced into a host cell. Such a nucleotide sequence may first be inserted into an appropriate recombinant expression vector as previously described herein.

The vectors in this aspect of the invention may include other known genetic elements necessary or desirable for efficient expression of the nucleic acid sequence from the RegIII protein promoter in a specified mammalian cell, including regulatory elements. For example, the vectors may include any necessary enhancer sequences that cooperate with the promoter in vivo, for example, to achieve in vivo transcription of the reporter gene. The methods of introducing the nucleotide sequence into a host cell are identical to that previously described for producing RegIII proteins.

A wide variety of host cells may be utilized in the methods of screening in the present invention. Exemplary host cells include, for example, Chinese hamster ovary, E. coli, COS and Bacillus.

Alternatively, the nucleotide sequence encoding all or a portion of a RegIII protein may be utilized in the vector for the screening methods described herein. In such a case, the RegIII protein may be isolated and purified by techniques well known to the skilled artisan, including chromatographic, electrophoretic and centrifugation techniques, as previously described herein. Additionally, the RegIII protein may be quantified by methods known to the art.

After contacting a nucleotide sequence encoding a reporter gene, or a RegIII protein gene, operably linked to a RegIII protein promoter with a test agent thought to be effective in inhibiting production of a RegIII protein, it is determined if the test agent inhibits production of the reporter gene product. This endpoint may be determined by quantifying either the amount or activity of the reporter gene product. The method of quantification will depend on the reporter gene that is used, but may involve use of an enzyme-linked immunosorbent assay with antibodies to the reporter gene product. Additionally, the assay may measure chemiluminescence, fluorescence or radioactive decay, or other methods known in the art. Assays for determining the activity or amount of the reporter gene products described herein are known to the art. If the test agent inhibits production of the reporter gene product, it is classified as an agent for treating asthma.

The screening methods of the invention are performed either in vitro (for example by monitoring the activity or expression of a RegIII protein in a cell-based assay or in an enzymatic activity assay) or in vivo (for example by monitoring the activity or expression of a RegIII protein in tissue samples such as BAL after administering a test agent to a mammal). Exemplary mammals include without limitation human, mouse, rat and dog.

The invention also provides methods for treating asthma. “Treatment”, “treating” or “treated” as used herein, means preventing, reducing or eliminating at least one symptom or complication of asthma. Exemplary symptoms and/or complications of asthma include, but are not limited to, AHR, mucus hyperproduction, elevated serum IgE levels, elevated airway eosinophilia and airway remodeling. These methods include administering to a mammal, e.g., a human, in need thereof a therapeutic amount of an agent that decreases the production or activity of a RegIII protein. Other nonlimiting examples of mammals that can be treated with the agents of the invention include mouse, rat, and dog. A “therapeutic amount” represents an amount of an agent that is capable of inhibiting or decreasing the activity or production or expression of a RegIII protein and causes a clinically significant response. The clinical response includes an improvement in the condition treated or in the prevention of the condition. The particular dose of the agent administered according to this invention will, of course, be determined by the particular circumstances surrounding the case, including the agent administered, the particular asthma being treated and similar conditions. Agents that decrease the activity or production or expression of a RegIII protein include those agents discovered in the screening assays described herein. Additional agents, or inhibitors, are well known in the art and include, for example, proteins and peptides such as hormones, cytokines and antibodies and portions thereof, nucleic acids such as antisense oligonucleotides, siRNA, ribozymes, and aptamers, and small molecules. See, e.g., Houston, et al., PNAS 99(14): 9127-9132 (2002). By way of example, an antibody against a RegIII protein as used herein may be, without limitation, a polyclonal antibody, a monoclonal antibody, a chimeric antibody, a humanized antibody, a genetically engineered antibody, a bispecific antibody, antibody fragments (including but not limited to “Fv,” “F(ab′)₂,” “F(ab),” and “Dab”) and single chains representing the reactive portion of the antibody. Such an antibody includes antibodies belonging to any of the immunoglobulin classes, such as IgM, IgG, IgD, IgE, IgA or their subclasses or mixtures thereof. The invention further includes derivates of these antibodies, such as those that retain their FoxM1-binding activity while altering one or more other properties related to their use as a pharmaceutical agent, e.g., serum stability or efficiency of production. Nonlimiting examples of antibodies include anti-RegIII antibodies, anti-IL-13 neutralizing antibodies, anti-IL-13R and anti-IL-4R antibodies. Methods for production of each of the above antibody forms are well known to the art.

In various embodiments, such an antibody binds to a RegIII protein (e.g., hHIP, hPAP, RegIIIγ), IL-13, IL13-R, IL-4R, or another component of the RegIII signal pathway. In additional embodiments, the antibody binds an inhibitor of RegIII activity or expression. Methods for production of each of the above antibody forms are well known to the art.

Cells that can be used to synthesize antibodies include animal, fungal, bacterial cells or yeast cells after transformation. By way of nonlimiting example, hybridoma cells can be produced in a known manner from animals immunized with a RegIII protein and isolation of their antibody-producing B cells, selecting these cells for RegIII or IL-13 binding antibodies and subsequently fusing these cells to, for example, human or animal, for example, mouse mylenoma cells, human lymphoblastoid cells or heterohybridoma cells or by infecting these cells with appropriate viruses to produce immortalized cell lines.

By way of nonlimiting example, human RegIII (e.g., HIP, PAP) or IL-13 monoclonal antibodies may be used to detect a RegIII protein or treating a subject with asthma-like symptoms. The term “monoclonal” indicates that the character of the antibody obtained is from a substantially homogeneous population of antibodies (i.e., the individual antibodies comprising the population are identical except for possible naturally occurring mutations that may be present in minor amounts), and is not to be construed as requiring production of the antibody by any particular method.

Thus, the detection or quantification of a RegIII protein in a sample can be carried out by an immunoassay utilizing the specific binding reaction between the monoclonal antibody and the RegIII protein or IL-13. Various immunoassays are well known in the art and any of them can be employed. Examples of the immunoassays include sandwich methods employing the monoclonal antibody and another monoclonal antibody as primary and secondary antibodies, respectively, sandwich methods employing the monoclonal antibody and a polyclonal antibody as primary and secondary antibodies, staining methods employing gold colloid, agglutination methods, latex methods and chemical luminescence.

Antibody fragments may also be used for a RegIII protein or IL-13 detection of asthma treatment. Antibody fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. These antibodies or fragments thereof can be used alone or in mixtures. It is understood by those of skill in the art that antibodies against the IL-13 receptor or the IL-4 receptor are also useful as measures of treating asthma by decreasing activity or production or expression of a RegIII protein.

The invention includes a method for treating asthma that includes administering a therapeutic amount of an IL-13 antagonist wherein such administration decreases the production of a RegIII protein. As explained elsewhere herein, expression of a RegIII protein is regulated at least in part through the IL-13 pathway. In one non-limiting embodiment, the test agent is an antagonist of IL-13 that disrupts the ability of IL-13 to bind to the IL-13 receptor, resulting in the down-regulation of a RegIII protein. In one non-limiting embodiment, an antagonist of IL-13 blocks the ability of IL-13 from binding to the IL-13 receptor. In one example, the antagonist competes with the IL-13 receptor for binding IL-13. In one example, antagonists include, without limit, fusion proteins that comprise one or more binding chains of the IL-13 receptor. Nonlimiting examples of soluble fusion proteins include IL-13Rα1-Fc and IL-13Rα2-Fc. Exemplary test agents also include without limitation chemicals, including synthetic small molecules, nucleic acids such as antisense oligonucleotides, siRNA, ribozymes, and aptamers, peptides and proteins such as hormones, cytokines and antibodies or portions thereof, such as anti-IL13 neutralizing antibodies and anti-IL-13R and IL-4R antibodies.

The invention includes a method for treating asthma by administering a nucleic acid. Exemplary nucleic acids include, but are not limited to, a deoxyribonucleic acid or a ribonucleic acid. In one embodiment, the ribonucleic acid has a nucleotide sequence that is complementary to a portion of the nucleotide sequence set forth in SEQ ED NO:1 or SEQ ID NO:3, as set forth in FIGS. 1 and 3, encoding RegIII protein.

In another embodiment, small interfering RNAs (i.e., through the process of RNA interference) are used as inhibitors of a RegIII protein. RNA interference relates to sequence-specific, posttranscriptional gene silencing brought about by double-stranded RNA that is homologous to the silenced gene target. Methods for inhibiting production of a protein utilizing small interfering RNAs are well known to the art, and disclosed in, for example, PCT International Application Numbers WO 01/75164; WO 00/63364; WO 01/92513; WO 00/44895; and WO 99/32619. RNA interference constructs or siRNA duplex RNA molecules can be used to interfere with expression of a RegIII protein or IL-13. Typically at least 19, 21, 22, or 23 nucleotides of a RegIII protein or IL-13 are sufficient for a siRNA molecule. In one embodiment, a siRNA molecule has a 2-nucleotide 3′ overhang. If the siRNA is expressed in a cell from a construct, for example, from a hairpin molecule or from an inverted repeat of the desired RegIII or IL-13 sequence, then the endogenous cellular machinery will create the overhangs. The siRNA molecules can also be prepared by chemical synthesis, in vitro transcription, or digestion of long dsRNA by Rnase III or Dicer. Brummelkamp, et al., Science, 296:550-53 (2002); Elbashir, et al., Nature, 411:494-98 (2001); Elbashir, et al., Genes Dev, 15:188-200 (2001).

In one embodiment, the invention includes a method for treating asthma that includes co-administration of one or more inhibitors of the activity or production of a RegIII protein or expression and one or more antagonists of IL-13. Exemplary RegIII inhibitors include those that modulate (e.g., inhibit) the activity of a RegIII protein such as anti-RegIII antibodies, as well as those that modulate (e.g., inhibit) transcription of RegIII mRNA such as siRNA, ribozymes, aptamers, and antisense oligonucleotides. Exemplary antagonists of IL-13 include agents that block the ability of IL-13 to bind to the IL-13 receptor. Such agents include those that act on the IL-13 molecule such as sIL-13Rα1-Fc, sIL-13Rα2-Fc and anti IL-13 neutralizing antibodies as well as agents that act on the IL-13 receptor such as anti IL-13R antibodies and synthetic small molecules. Antagonists of IL-13 further include ribozymes, aptamers, siRNA, antisense oligonucleotides, cytokines, and hormones. Combination therapies involving agents that inhibit a RegIII protein and antagonize IL-13 are contemplated herein.

The invention includes methods for decreasing the expression of a RegIII protein in a mammal. The method includes administering an effective amount of an antagonist of IL-13. In one embodiment, the antagonist blocks the ability of IL-13 to bind to the IL-13 receptor. In another embodiment, the antagonist blocks the ability of IL-13 to bind to the IL-4 receptor. Such antagonists include those discussed above in connection with methods of treating asthma. In certain embodiments the antagonist is selected from the group comprising sIL-13Rα1-Fc, sIL-13Rα2-Fc and neutralizing antibodies to IL-13.

In another aspect, the invention includes methods for monitoring the efficacy of a treatment for asthma. The methods include administering a test agent and monitoring the expression of a RegIII protein, wherein a decrease in the expression of a RegIII protein indicates that the test agent is useful in treating asthma. Test agents include those described elsewhere herein. In one embodiment, the method includes administering an antagonist of IL-13. Antagonists of IL-13 include those described above. Non-limiting examples of IL-13 antagonists include sIL-13Rα1-Fc, sIL13Rα2-c, and neutralizing antibodies to IL-13.

The methods for treating asthma include administering to a mammal in need thereof an IL-13 antagonist, detecting a level of activity or expression or production of a RegIII protein and comparing the level of activity or expression of a RegIII protein to a control level prior to treatment with the IL-13 antagonist. The level of activity or expression or production of a RegIII protein may be increased or decreased relative to the control level. If the test agent reduces or inhibits the activity or expression of a RegIII protein, then it may be classified as an IL-13 antagonist that is efficacious for treating asthma. Exemplary agents that inhibit the activity or expression of RegIII include, without limitation, sIL-13Rα1-Fc, sIL13Rα2-c, neutralizing antibodies to IL-13 and anti-RegIII antibodies

The agents may be administered by a wide variety of routes. Exemplary routes of administration include oral, parenteral, transdermal, and pulmonary administration. For example, the agents may be administered intranasally, intramuscularly, subcutaneously, intraperitoneally, intravaginally and any combination thereof. For pulmonary administration nebulizers, inhalers or aerosol dispensers may be used to deliver the therapeutic agent in an appropriate formulation (i.e., with an aerolizing agent). In addition, the agents may be administered alone or in combination with other agents or known drugs. In combination, agents may be administered simultaneously or each agent may be administered at different times. When combined with one or more known asthma drugs, agents and drugs may be administered simultaneously or the agent can be administered before or after the drug(s).

In one embodiment, the agents are administered in a pharmaceutically acceptable carrier. Any suitable carrier known in the art may be used. In one embodiment, the carriers efficiently solubilize the agents. Carriers include, but are not limited to a solid, liquid or a mixture of a solid and a liquid. The carriers may take the form of capsules, tablets, pills, powders, lozenges, suspensions, emulsions or syrups. The carriers may include substances that act as flavoring agents, lubricants, solubilizers, suspending agents, binders, stabilizers, tablet disintegrating agents and encapsulating materials.

Tablets for systemic oral administration may include excipients, as known in the art, such as calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with disintegrating agents, such as maize, starch or alginic acid, binding agents, such as gelatin, collagen or acacia and lubricating agents, such as magnesium stearate, stearic acid or talc.

In powders, the carrier is a finely divided solid, which is mixed with an effective amount of a finely divided agent.

In solutions, suspensions or syrups, an effective amount of the agent is dissolved or suspended in a carrier such as sterile water or an organic solvent, such as aqueous propylene glycol. Other compositions can be made by dispersing the inhibitor in an aqueous starch or sodium carboxymethyl cellulose solution or a suitable oil known to the art.

The agents are administered in a therapeutic amount. Such an amount is effective in treating asthma. This amount may vary, depending on the activity of the agent utilized, the nature of the asthma and the health of the patient. The term “therapeutically effective amount” is used to denote treatments at dosages effective to achieve the therapeutic result sought. Furthermore, a skilled practitioner will appreciate that the therapeutically effective amount of the agent may be lowered or increased by fine tuning and/or by administering more than one agent, or by administering an agent with an anti-asthmatic compound (e.g., corticosteroid). As illustrated in the following examples, therapeutically effective amounts may be easily determined, for example, empirically by starting at relatively low amounts and by step-wise increments with concurrent evaluation of beneficial effect. (i.e., reduction in asthmatic symptoms following exposure to antigen).

When the agents are combined with a carrier, they may be present in an amount of about 1 weight percent to about 99 weight percent, the remainder being composed of the pharmaceutically acceptable carrier. In some embodiments, the agents are present in an amount of about 25 weight percent to about 75 weight percent. In some embodiments, the agents are present in an amount of about 30 weight percent to about 60 weight percent. In some embodiments, the agents are present in an amount of about 40 weight percent to about 50 weight percent.

Reference will now be made to specific examples illustrating the invention. It is to be understood that the examples are provided to illustrate embodiments of the invention and that no limitation to the scope of the invention is intended thereby.

### EXAMPLE 1

### Gene Expression Changes in Mouse Lung Associated with Allergic Reaction

To identify the gene expression changes induced by intratracheal OVA-challenge Balb/C mice (Jackson Laboratories (Bar Harbor, Me.)) were immunized by an intraperitoneal (i.p.) injection of 10 μg of ovalbumin (OVA) (Sigma, St. Louis, Mo.) in 200 μl of PBS on day 0. On days 14 and 25, mice were anesthetized with a mixture of ketamine and xylazine (45 and 8 mg/kg respectively) and challenged intratracheally with 50 μl of a 1.5% solution of OVA or an equivalent volume of PBS. To identify changes in mRNA concentration dependent on IL-13 mediated signal transduction, two of the OVA-challenged mice were treated with three intraperitoneal injections of the soluble IL-13 receptor fusion protein, sIL-13Rα2-Fc, prior to and during the course of the allergic challenge. As control for the Fc-moiety of the receptor fusion protein, two of the OVA-challenged mice were similarly treated with intraperitoneal administration of hIgG. A second set of six control mice were similarly sensitized to OVA without subsequent challenge and treated on an identical time course with intratracheal administration of PBS buffer, either alone (n=2) or with intraperitoneal injection of hIgG (n=2) or sIL-13Rα2-Fc (n=2). Lung tissue for the OVA-challenged and buffer-alone control mice was harvested at 78 hr following the second pulmonary antigen challenge (day 28).

Snap frozen mouse lung tissue was pulverized using liquid nitrogen chilled mortar and pestle, suspended in 6 ml 4M guanidinium isothiocyanate/0.7% 2-mercaptoethanol (GTC/ME) and pulse sonicated for 2 minutes. The tissue suspension was extracted twice with acid equilibrated phenol (Promega Total RNA Kit) and nucleic acid precipitated with an equal volume of isopropanol. The pellet was resuspended in 0.8 ml GTC/ME, reextracted twice with an equal volume of acid phenol and once with chloroform. RNA was ethanol-precipitated, suspended in DEPC treated H2O and quantified by OD280.

cDNA was synthesized from 10 μg of total RNA using the Superscript Kit (BRL) with modifications described in Byrne, et al., “Preparation of mRNA for expression monitoring,” Current Protocols in Molecular Biology John Wiley and Sons, Inc. (New York 2000). First strand synthesis was carried out at 50° C. to prevent miswriting from ribosomal RNA and utilized a T7 RNA polymerase promoter containing poly-T primer (T7T24) for subsequent in vitro antisepses RNA (crank) amplification and biotin labeling. cDNA was purified using Biome Carboxyterminated beads (Polysciences) according to manufacturer's instructions, and eluted in 48 μl of 10 mM NaAcetate pH 7.8.

In vitro T7 polymerase driven transcription reactions for synthesis and biotin labeling of antisense cRNA, Qiagen RNeasy spin column purification and cRNA fragmentation were carried out. GeneChip® hybridization mixtures contained 10 μg fragmented cRNA, 0.5 mg/ml acetylated BSA, 0.1 mg/ml herring sperm DNA, in 1×MES buffer in a total volume of 200 μl as per manufacturer's instructions. Reaction mixtures were hybridized for 18 hours at 45° C. to Affymetrix Mu11KsubA and Mu11KsubB oligonucleotide arrays. The hybridization mixtures were removed and the arrays were washed and stained with Streptavidin R-phycoerythrin (Molecular Probes) using the GeneChip® Fluidics Station 400 and scanned with a Hewlett Packard GeneArray Scanner following manufacturer's instructions. Fluorescent data was collected and converted to gene specific difference averages using MicroArray Suite 4.0 software.

An eleven-member standard curve was prepared by spiking gene fragments derived from cloned bacterial and bacteriophage sequences into each hybridization mixture at concentrations ranging from 0.5 pM to 150 pM. These standards represented RNA frequencies of approximately 3.3 to 1000 parts per million (ppm) assuming an average transcript size of 2 kb. The biotinylated standard curve fragments were synthesized by T7 polymerase driven IVT reactions from plasmid-based templates. The spiked biotinylated RNA fragments serve both as an internal standard to assess chip sensitivity and as standard curve to convert measured fluorescent difference averages from individual genes into RNA frequencies in ppm. Average fluorescence difference between perfect match and single mismatch probe sets containing gene-specific oligonucleotides were used to determine frequency values with respect to spiked standard curve. In addition, a second set of algorithms based primarily on the fraction of individual positive or negative responding probe pairs, is used to assess the absolute presence or absence of the gene product. The sensitivity of the individual microarray chip is set at one-half the minimum concentration at which 2 or any 3 adjacent standard curve spike-in templates are called present. The standard curve linear regression is forced through zero and the minimum reported gene frequency is set to the sensitivity of the individual GeneChip®.

Multiple independent replicas for each of the treatment or control experimental conditions were measured and the expression data subjected to routine statistical analysis in an effort to remove false positives. Frequency values determined from individual measurements for a given experimental set were initially compared. Average values for treatment and control animals were compared to obtain average fold change (AFC). Two tailed Student t-tests were calculated using either unequal covariance with raw frequency values or equal covariance with log-transformed frequency values.

The overall gene expression measured for each of the three treatment groups used to identify allergen-challenge induced gene expression was well balanced with respect to mRNA integrity, number of genes called present and total mRNA frequency computed across the various control and treatment files (data not shown).

The gene expression profile measured for control mice treated with PBS was not significantly altered by intraperitoneal co-administration of human IgG or sIL-13Rα2-Fc, and thus frequency values from the six control mice were combined as a single set in the calculation of average untreated baseline expression values. Similarly, the four OVA-challenged mice treated either with intraperitoneal co-administration of buffer or hIgG were combined as a single set in calculation of average frequency values for pulmonary allergen-challenged mRNA frequency. The data are shown on Table 2 below.

TABLE 2
Frequency values for RegIIIγ mRNA following allergen challenge

| | mRNA Frequency |
|---|---|
| Sample | (ppm ± SD) |
| PBS Control | 114.2 |
| OVA | 228.0 |
| OVA and sIL-13Rα2-Fc | 67.5 |

The data demonstrate that RegIIIγ protein mRNA is specifically induced by direct pulmonary intratracheal administration of IL-13 or ovalbumin-induced allergic challenge. Additionally, these data show that inhibition of IL-13 activating using the soluble receptor antagonist (sIL-13Rα2-Fc) completely inhibits the expression of RegIIIγ protein by ovalbumin challenge. Physiological studies utilizing the sIL-13Rα2-Fc antagonist have previously shown IL-13 activity is essential to asthma disease pathology, including epithelial mucus production and AHR. Thus, RegIIIγ protein is identified as an IL-13 responsive gene downstream of the ovalbumin allergic challenge, which identifies it as a therapeutic agent in the target of asthma.

These data confirm that intraperitoneal administration of the soluble IL-13 antagonist was able to completely inhibit the induction of OVA allergic pulmonary RegIIIγ protein. Physiological studies utilizing the sIL-13Rα2-Fc antagonist has previously shown to be IL-13 activity essential for asthma disease pathology including epithelial mucus production and AHR. Wills-Karp, M, et al., Science 282(5397):2258-61 (1998). Thus, these data demonstrate that therapeutic intervention resulting in the inhibition of RegIIIγ protein is associated with a decrease of asthma related symptoms.

### EXAMPLE 2

### Gene Expression Changes in Mouse Lung Induced by mIL-13 Lung Instillation

To identify IL-13 mediated changes in pulmonary gene expression, six Balb/C mice (Jackson Laboratories, Bar Harbor, Me.) were treated with multiple 5 μg dose (0, 24 hr, and 48 hr) lung instillation of recombinant mIL-13. A second set of control Balb/C mice (n=4) were instilled with buffer alone on an identical schedule. Additionally, a set of Stat6−/− null mice were treated identically with multiple doses mIL13 (n=4) or PBS buffer (n=5) lung instillation prior to harvesting of all lungs at 78 hr for expression profiling. Stat6−/− is an additional control; it is a key intermediate in IL-13 signaling pathway, critical for mucus production and AHR; the absence of this IL-13 signaling transducer ameliorates asthmatic symptoms. The overall gene expression for each of the three treatment groups used to identify allergen-challenge induced gene expression was well-balanced with respect to mRNA integrity, number of genes called present; and total mRNA frequency computed across the various control and treatment files. The data are shown in Table 3.

TABLE 3

| | | RegIIIγ protein |
|---|---|---|
| Animal | Treatment | mRNA Frequency (ppm ± SD) |
| Balb/C | PBS | 43.8 |
| | rIL-13 | 370.0 |
| Stat6^{-/-} | PBS | 61.5 |
| | rIL-13 | 131.0 |

These data confirm that increases in mRNA concentration are mediated by mIL-13 lung instillation.

### EXAMPLE 5

### Prophetic Example of Screening Assay for Inhibitor of a RegIII Protein Activity

A human RegIII protein (e.g., hHIP, hPAP) is cloned into bacterial expression vector, transformed into E. coli or COS cells and is purified from bacterial or mammalian cultures by column chromatography utilizing standard molecular biology and biochemistry methods. Primary epithelial cells or epithelial cell lines are then treated with the RegIII protein. After contact with the protein, the cells or cell lines are assayed for an increase in epithelial cells or mucin. Test agents are screened by their ability to modulate (e.g., inhibit) the mucus production and/or proliferation of epithelial cells as determined by staining of the epithelial cells or by microscopic observation of increased secretory granuoles.

### EXAMPLE 6

### Prophetic Example of Screening Assay for Inhibitor of a RegIII Protein Production Involving a RegIII Protein Promoter

A protein promoter of a RegIII protein is linked to a reporter gene, for example, a luciferase. Activation of the reporter gene is demonstrated by inducing with IL-13, indicating transcriptional specificity. Test agents are screened to identify those that block the IL-13 inducted reporter gene activity.

### EXAMPLE 7

### Treating Asthma with a RegIII Protein Inhibitor

A therapeutically effective amount of a known protein inhibitor of a RegIII protein is administered to a subject diagnosed with asthma. A control group also exhibiting asthmatic symptoms is treated will a placebo control. Administration may be by a single treatment or treatment over a course of days. Subjects are evaluated for asthma-related symptoms, such as AHR and mucus production. Effective treatment is determined by a reduction in asthma-related symptoms compared to the control group.

While the invention has been illustrated and described in detail in the drawings and foregoing description, the same is to be considered as illustrative and not restrictive in character, it being understood that only certain embodiments have been shown and described and that all changes and modifications that come within the spirit of the invention are desired to be protected. In addition, all references cited herein are indicative of the level of skill in the art and are hereby incorporated by reference in their entirety.

What is claimed is:

1. A method of screening for agents for treating asthma in a mammal, comprising:
(a) contacting a RegIII protein with a test agent; and
(b) determining if the test agent inhibits the activity of the RegIII protein, wherein a test agent that inactivates the activity of the RegIII protein is useful for treating asthma.

2. The method of claim 1, wherein the mammal is a human.

3. The method of claim 1, wherein the asthma is selected from the group consisting of atopic asthma, nonatopic asthma, allergic asthma, exercise-induced asthma, drug-induced asthma, occupational asthma, and late stage asthma.

4. The method of claim 1, wherein the determining if the test agent inhibits the activity of the RegIII protein comprises performing a cell proliferation assay.

5. The method of claim 4, wherein the cell proliferation assay is selected from the group consisting of a labeled thymidine uptake assay, a 5-bromo-2′-deoxyuridine uptake assay, a 3-[4,5-dimethylthiazol-2-yl]-2,5-diphenyltetrasodium bromide assay, an adenosine triphosphate generation assay and a combination thereof.

6. The method of claim 4, wherein the cells are selected from the group consisting of primary epithelial cells or epithelial cell lines.

7. The method of claim 1, wherein the RegIII protein is a mammalian RegIII protein.

8. The method of claim 1, wherein the RegIII protein has an amino acid sequence selected from the group consisting of SEQ ID NO:2 and SEQ ID NO:4.

9. The method of claim 1, wherein the RegIII protein has an amino acid sequence having at least about 70% identity to an amino acid sequence selected from the group consisting of SEQ ID NO:2 and SEQ ID NO:4.

10. A method of screening for agents for treating asthma in a mammal, comprising:
(a) contacting a nucleotide sequence encoding a reporter gene product operably linked to a mammalian RegIII protein promoter with a test agent; and
(b) determining if the test agent inhibits production of the reporter gene product, wherein a test agent that inactivates the activity of the RegIII protein is useful for treating asthma.

11. The method of claim 10, wherein the mammal is a human.

12. The method of claim 10, wherein the asthma is selected from the group consisting of atopic asthma, nonatopic asthma, allergic asthma, exercise-induced asthma, drug-induced asthma, occupational asthma, and late stage asthma.

13. The method of claim 10, wherein determining if the test agent inhibits production of the RegIII protein comprises quantifying the amount or activity of the reporter gene product.

14. The method of claim 10, wherein the RegIII protein promoter has a nucleotide sequence of SEQ ID NO:5.

15. The method of claim 10, wherein the RegIIIγ protein promoter has a nucleotide sequence having at least about 80% identity to the nucleotide sequence of SEQ ID NO:5.

16. The method of claim 10, wherein the RegIII protein promoter has a nucleotide sequence of SEQ ID NO:6.

17. The method of claim 10, wherein the RegIII protein promoter has a nucleotide sequence having at least about 80% identity to the nucleotide sequence of SEQ ID NO:6.

18. The method of claim 10, wherein the reporter gene product is selected from the group consisting of luciferase, β-galactosidase, chloramphenical acetyltransferase, β-glucuronidase, alkaline phosphatase, and green fluorescent protein.

19. A method for treating asthma, comprising administering to a mammal in need thereof a therapeutic amount of an agent that decreases the activity of a mammalian RegIIIγ protein.

20. The method of claim 19, wherein the mammal is a human.

21. The method of claim 19, wherein treating the asthma is performed by a method selected from the group consisting of decreasing AHR, decreasing mucus hyperproduction, decreasing serum IgE levels, and decreasing airway eosinophilia.

22. The method of claim 19, wherein the agent that decreases the activity of the mammalian RegIII protein is sIL-13Rα2-Fc.

23. The method of claim 19, wherein the mammalian RegIII protein is encoded by a nucleotide sequence selected from the group consisting of SEQ ID NO:1 and SEQ ID NO:3.

24. The method of claim 19, wherein the RegIII protein is encoded by a nucleotide sequence having at least about 70% identity to a nucleotide sequence selected from the group consisting of SEQ ID NO:1 and SEQ ID NO:3.

25. A method for treating asthma, comprising administering to a mammal in need thereof a therapeutic amount of an agent that decreases the production of a mammalian RegIII protein.

26. The method of claim 25, wherein the agent that decreases the production of the RegIII protein is a nucleic acid.

27. The method of claim 26, wherein the nucleic acid is a ribonucleic acid.

28. The method of claim 27, wherein the ribonucleic acid has a nucleotide sequence that is complementary to a portion of the nucleotide sequence set forth in SEQ ID NO:1 or SEQ ID NO:3 encoding the RegIII protein.

29. The method of claim 27, wherein the ribonucleic acid is RNA interference.

30. The method of claim 25, wherein the mammal is a human.

31. The method of claim 25, wherein the inhibitor is administered in a pharmaceutically acceptable carrier.


SEQUENCE LISTING (6 sequences)

SEQ ID NO: 1 - DNA, 807 bp, Homo sapiens
aaaccataccatatcccaccagagagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 2 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 3 - DNA, 774 bp, Mus musculus
acaccatcctagggatctgcaagacagacaagatgcttccccgtataaccatcaccatcatgtcctggatgctgctctcctgcctgatgctcctttctcaggtgcaaggtgaagttgccaagaaagatgccccatcttcacgtagcagctgccccaagggctcccgtgcctatggctcctattgctatgccttgtttagtgtatctaaaaactggtatgatgcagatatggcctgccaaaagaggccctcaggacatcttgtgtctgtgctcagtggcgctgaagcttccttcctgtcctccatgatcaaaagcagtggtaacagtggccaatatgtatggattgggctccatgacccgacactgggctatgaacccaacagaggtggatgggagtggagcaatgctgatgtgatgaattacatcaactgggagacgaatccttcctcttcctcaggcaatcactgtggtaccctgtcaagagcctcaggatttctgaagtggagagagaattattgtaacttagagttaccctatgtctgcaaattcaaggcctagagtacagaattgacatcatgggttgatatatacctagccacaagcaagatcccaaatgtgaagacagatgaaaaagggaacattctccccacaatcgaaatctgacctcctgattttctccttctctggcccttccctcctctcatttgaggaactctgtgtgcactatggccttaattctcaaagcataatattaaagtatataactcatgctcat

SEQ ID NO: 4 - protein, 174 aa, Mus musculus
MLPRITITIMSWMLLSCLMLLSQVQGEVAKKDAPSSRSSCPKGSRAYGSYCYALFSVSKNWYDADMACQKRPSGHLVSVLSGAEASFLSSMIKSSGNSGQYVWIGLHDPTLGYEPNRGGWEWSNADVMNYINWETNPSSSSGNHCGTLSRASGFLKWRENYCNLELPYVCKFKA

SEQ ID NO: 5 - DNA, 2100 bp, Homo sapiens
gaattcctgtgtcatccctaagaagtagatttttatataactcaaaaccagctcaagaaatataatatttaaacaaacttctcagtgcttggttaataagcattgttatgaagaaacaatgaaaaataaaacaaaaacaactcaactttagtgagtttagtgagtgtcattgcaaagaaaatttcacattgcacttagggattttataaccttaaggcttattacaaaggtcacactatttcaattaagaacaaaagtgagttttaataataaactgtcagcatttcaaatcttctctcctaggtatcttgaaatatacaacatattgttgttaactatagtcaccctatgtgctatcaaaagctagaacatattcctcctatctagctgtatgtttgtacgatgaactaaactcttttcatctcccctcacccgtctcagcctctggtaactattctaccctctggttaccttctaccccgtacctccatgtgtttacatattgttgtataaattttgtggaaaaaaatcctttttaacaaaataatctgctggctatatttcttctcatattatttgaggaattgcattgaattttgggtactgaattttgaggacgtgaattaaggatgagctacatctcatgacctcgtcacagacccctactataaacatctgcatacaagttggcatattacaacaatagcatgagagtcagggtctgtagaaattcccagcctaactttttcctggactggccaaactgaacctgaaccagtgtatgggcaatgtagtatgcttgtcagaggcaccaaactgctaactcaaaaataaacatgactaaaagaacttctcccagtatacccatatgaagctgaccatttgcccagtaagaatgcaatgaaaagggcttttaatgtacagccaaatggcagtccagcaaggaagctcatgagcaacctacaagtagtgagcattttgatgtaatagagacttacatgcaattcagaattatgtttacaacatcacaaaacaaaaatttagagataggctctcactctgttgcctaagctggagtgcagtgggatgatcataacttactgcagccttgaactcctgggttcaactgaaggtcctcctacctcagcctgctgagtagctaggaccacaagcacacaccaccgcaactggcttaaattaaaatataaattgtagagatagggtcttaatgtgttgcccaggctgctcttgaactccttgcttcaggtgatcctcccacctcagcctctcaaagtgctgggattatagacctgagccacagcacctggccaactgacctatgattttacacaatggctgctcttcccttctttaactattattcattcttctttgatcctcattatttgactgtagtccttcttatgtcttgttttccttcattacctcttattctatcacattgccattgtcattctccactggggaagctctttcttgctgaagactggaaagacaagtccattcacctgattttctgtaagattgtggctcatgtattgacttgtcagacaattctgaagtttcatcaaaattagctatcatgcttgcataatggccctgaaccctcactcctactcttagcttcagtaccatctatgtcctcaactgtccatgatacttataattcccgtaaatcttcacttaacacctaacatttatttaatcttactaggcaaggtaataagaaatacataggtttgcctccagaagtgggttcttaagaaacccaccagaggaactcctctttcagatgtccacattagaagatttcatatcacatttggtgccacaggcctttgacaaggaggatgcagaggaaaaagcaaacttcacctcttcctagggaaagtgttggcctgccaacaggaaagaggcaacatctgggaaaatccccagtctttgccaggaagagtccatgccaaccccaccccatgacccctgtcctgcctactcattgtcactcttcactccaatgtccctcccccagatcctcttataaaatcccactctttcctgaccagacaaaccataccatat

SEQ ID NO: 6 - DNA, 2000 bp, Mus musculus
atctaaatgtatattacatagtgggattcacatatatttccctcaaatattgaatattttgttatattattttcttaattaattgtttgaattcacagttttttattttagtggttgcatctattttgatacaatattaaatatattaatatctaactttctggaaattaatatacaaacaataattacaatttctcaaattactttcctcttagaaatctttatatattcagttattctcaaagcatcaatattattcacatcattagtgtttatagcactagacatttacaggcagaagcctggcatatctgctctctatccagaagttgactgaaacatatgcagatacccgtagccaaatattaagcagaggtcagagacccatatggaagagttaggggaagggtggaaggcctggaaggagaaagaaaatccacaggtacaataatatgtcaactaaactggaactttgggagctctcagagactgagccacaacccaaagagtatatccaggctggacagaggcccctggtacatgtgtagcagaggactgatttgtctggccaccgtgggagaggatgtgcctaatcctataggcacttgatgtgccagggttggggataccctgggagacccatcctctcagaagcaaaagaagggggaatgagggagagagtctgggtggagaggcacagagtggggcagcatttggaatgcaaataaataaataaatgcataaataaataacaaaaaataccaattatatttgaacatttttaaagcatgttgtaaaaatgtgcatatattttgaaaaccatatgtgtgtcaaatcattttagccttgctttttttcaggacagcaattagtcttcagggagaaacctagtgttttaaagtattgaaacaatcaagtttgttaacgttaattatattgtttgtttgatgtagttcaaggtcattctaatgtgaaacgtgaactcctttctgcctgaacatgcatgggtgatggacacaagttaaagactctggatggattaatgctaaagcacacatcatttaggtacaatttccttaacctcatgaacacaagtgttggtaccattaaaatatcacgtacagatttagcatagttatgaaaactacgtcaaagccattctaagggattttaaaagacctcacaaatgtctgatcaaagtttgagaataggacactgaatatcaagtaaacaattcagaattacagtaaatcaaggccttgtttctcaactttcccacagagaggcagcaggaaagcaagtccattctaccactgttacttgaaacctcagctaaaatattgactcaaaagattattatcaaggttcctgtaagtcttggtacaaagttcagtttttctcctttctcactttcctttctttcagctaattcacgaccatcaattgttataattcccaacttatcaaaaatctaatcactgatataaatattacaaaacaaagagccaagaaatatacatatgaatctacagttataaggtcttctttgcattgtgacaaaaataccctgttaatgtctgatttcttcagtaaactcaaggttgcaatattttaaaaatgttagcacagcattatctatagcataactattatttctataagttttccggaacatagtatctgggaaaagatggggaaagttcaaactgcacccatacctgacaattcagcagggctgctaacaggaaagggccacatatgtaacagtttatagtctttggcaggaagaaactcccgcttttgatgggaaacccctagagtccatgccaagcaccacctacctccctgacttgcatgagcattctgcttattcactttgatgcccctccctcaagtcctactataaaaacagtcatctctgtcctgacacaccatcctagggatctgcaagacaggtaagtaggaactttgtaggggttcaaccatcataccacaggaga